(19) United States
(12) Patent Application Publication
(10) Pub. No.: US 2014/0193514 A1
(43) Pub. Date: Jul. 10, 2014

(54) Method for the Treatment of Cancer

(71) Applicant: Mitchell S. Felder, Hermitage, PA (US)

(72) Inventor: Mitchell S. Felder, Hermitage, PA (US)

(73) Assignee: MARV ENTERPRISES, Hermitage, PA (US)

(21) Appl. No.: 14/238,185

(22) PCT Filed: Aug. 16, 2012
(86) PCT No.: PCT/US2012/051098

§ 371 Date: Feb. 10, 2014

Related U.S. Application Data
(60) Provisional application No. 61/525,464, filed on Aug. 19, 2011.

(51) Int. Cl. A61M 1/36; A61M 1/16

(52) U.S. Cl. CPC A61M 1/362; A61M 1/16; A61M 1/3618; A61M 1/3681
USPC 424/537; 210/663; 210/638; 210/662

(57) ABSTRACT

The invention describes a method to treat an extracorporeally body fluid. The body fluid can include, for example, blood, cerebral spinal fluid and lymph. A first stage of the method applies a treatment to the extracorporeal body fluid. The treatment comprises combining at least one antibody with a CA antigen to produce an antibody-CA antigen moiety. A second stage of the method substantially removes the antibody-CA antigen moiety from the extracorporeal body fluid.

The present invention is being submitted as a provisional application under 37 CFR 1.53(c).

### FIELD OF THE INVENTION

The invention relates to a device and method for the treatment of cancer using extracorporeal treatment of body fluid.

### BACKGROUND OF THE INVENTION

In the United States cancer is the second leading cause of death. Cancer has surpassed heart disease is the number one cause of dead) in patients younger than 85 years of age. In the United States each year 1.3 million cases of cancer are diagnosed and more than 570,000 people die each year from cancer. The highest mortality rates involving lung, colorectal, breast and prostate cancers. Each year, more Americans die from cancer than were killed in all of the wars in which the United States participated during the twentieth century. The average age at the time of diagnosis for cancer is 67 years, and about three-quarters of all cancers are diagnosed after the age of 55. Further, cancer is the second leading cause of death in children up to the age of fourteen. In children leukemia is the most common cause of death from cancer. Over 8 million Americans alive today have had some form of cancer. Approximately one in three Americans will develop some form of cancer within their lifetime.

The metastasis of cancer within a body is believed to be facilitated by the formation of new blood vessels supplying the cancer cells with nutrients. Angiogenesis is the physiological process of the growth of new blood vessels from pre-existing vessels. These new blood vessels supply nutrients to the cancer cells and facilitate growth of malignant tumors and the spread of cancer ceils to other parts of the body.

Certain molecular organic compounds are implicated as causing or allowing angiogenesis which in turn allows the metastasis of various cancer cells and growth of cancerous tumors.

### SUMMARY OF THE INVENTION

In general terms, the present invention relates to the treatment of cancers, hereinafter abbreviated as “CA”. Specifically, the invention pertains to a method for the extracorporeal treatment of one or more body fluids in two stages characterized by removing a body fluid from a living body diseased with a type of CA, passing the body fluid through a first stage; applying an anti-angiogenesis, anti-tumorigenesis, anti-metastasis, or chemotherapeutic treatment to at least one antigen in the body fluid. The body fluid can include, for example, blood, lymph or cerebrospinal fluid.

More specifically, the treatment comprises creating an antibody-antigen moiety in a first stage; passing the treated body fluid to a second stage; removing antibody-antigen moiety from the body fluid during passage through the second stage, and returning the purified body fluid to the body.

The invention is further characterized by targeting an antigen in the body fluid, with an antibody to allow and facilitate removal thereof in the second stage. Specifically, the method is further characterized by removing body fluid from a person to produce the extracorporeal, bodily fluid; imposing a treatment acting on an antigen of CA in the body fluid, filtering or otherwise removing the treatment from the body fluid, and returning the body fluid to the patient after removing substantially all of the treatment in the second stage.

The method of the present invention comprises extracorporeally treating at least one component of a patient's body fluid with a designer antibody containing an albumin-moiety which will create an albumin-antibody-CA antigen moiety allowing for the efficacious dialysis of the resultant albumin-antibody-CA antigen compound (the targeted CA antigen being respectively, one or a combination of antigen(s) from: CA Ant. Ang., CA Ant. T., CA Ant. ST, CA Ant. Sp., CA Ant Chem.).

More specifically, the method is characterized by removing body fluid from a person to produce the extracorporeal bodily fluid; directing a first antibody against the targeted CA antigen (CA Ant. Ang., CA Ant. T., CA Ant. ST, CA Ant. Sp., CA Ant. Chem.) in the first stage of extra-corporeal treatment in the body fluid; in the second stage directing a second antibody conjugated with albumin and/or a protein against the targeted CA antigen thereby farming an albumin-antibody-CA antigen compound; removing at least a substantial portion of the albumin-antibody-CA antigen compound from the body fluid by dialysis, other filtering, or other means; and returning the body fluid to the patient.

The method is also characterized by testing the blood and/or CSF to determine the efficacy of treatment before returning the body fluid to the patient.

### BRIEF DESCRIPTION OF THE DRAWINGS

FIG. 1 is a partial cross sectional view of a cylinder and tubing used to deliver a treatment to a bodily fluid.

FIG. 2 is a partial cross sectional view showing additional detail of the cylinder and tubing of FIG. 1.

### DETAILED DESCRIPTION OF THE INVENTION

U.S. Ser. No. 13/157,635 and PCT/US2010/027474 are hereby incorporated by reference. In the first stage of treatment, a selected body fluid is removed using a standard catheter and/or lumbar puncture, in the second stage, the body fluid is treated with antibodies against the targeted CA antigen (CA Ant. Ang., CA Ant. T., CA Ant. ST, CA Ant. Sp., CA. Ant. Chem.).

The method of the present invention comprises treating at least one component of a patient's body fluid extracorporeally with a designer antibody containing an albumin-moiety to create an albumin-antibody-CA antigen moiety allowing for the efficacious dialysis, filtering or other means of removal of the resultant albumin-antibody-CA antigen compound (the targeted CA antigen being respectively, one or a combination of antigen(s) from: CA Ant. Ang., CA Ant. T., CA Ant. ST, CA Ant. Sp., CA Ant. Chem.).

The targeted antigens would include one, or a combination of:
1. Antigens involved in causing or facilitating Angiogenesis (CA Ant. Ang.), including but not limited to:
   a. VEGF; Vascular endothelial growth factor;
   b. VEGFR: Vascular endothelial growth factor receptor tyrosine kinase inhibitor;
   c. NRP-1: Neurolipin-1;
   d. Ang1: Angiopoietin 1;
   e. Tie2: Tyrosine kinase/CD 202B (Cluster of differentiation 202B);
   f. PDGF-BB: platelet derived growth factor;
   g. Endoglin: CD105;
   h. TGF-beta: Transforming growth factor beta;
   i. FGF: Fibroblast growth factor;
   j. HGF: Hepatocyte growth factor/scatter factor;
   k. MCP-1: Monocyte chemotactic protein-1;
   l. Integrins; heterodimers with alpha and beta subunits;
   m. VE-cadherin: Vascular endothelial-cadherin; CD144 (Cluster of Differentiation 144), Cadherin 5, type 2;
   n. PECAM: Platelet Endothelial Cell Adhesion Molecule/CD 31 (Cluster of differentiation molecule);
   o. Matrix metalioproteinase: 2,3,7 and 9;
   p. PAI-1: Plasminogen Activator Inhibitor-1;
   q. CXC Chemokines;
   r. Id1/Id3: Inhibitors of differentiation;
2. Antigens involved in causing or facilitating Tumorigenesis; (CA Ant. T.), including but not limited to:
   a. IGF-1; insulin-like growth factor 1;
   b. IGF-2: insulin-like growth factor 2;
   c. FGF: Fibroblast growth factor;
   d. NGF: Nerve growth factor;
   e. PDGF: Platelet derived growth factor;
   f. Tumor growth factor; alpha and beta;
3. Antigens involved in a Signal Transducer; (CA Ant. ST), including but not limited to
   a. Sonic hedgehog homolog: (SHH);
   b. Indian hedgehog homolog: (IHH);
   c. COX2: Cyclooxygenase-2;
4. Antigens which are unique to specific CAs (examples); (CA Ant. Sp.) including but not limited to:
   a. MTA1: Metastasis associated protein 1 (breast cancer);
   b. AGR2; Anterior gradient 2 (adenocarcinomas of the pancreas, esophagus, prostate., lung cancer);
   c. Tau protein (breast cancer);
   d. GL12 (melanoma metastasis);
   e. Integral alpha3beta1 (breast cancer);
   f. CCL25 (Ovarian cancer);
   g. Kif18A (Breast cancer);
   h. MMP9 (Nasopharyngeal carcinoma);
   i. Type 1 gamma phosphatidylinositol phosphate kinase (Breast cancer);
   j. Ubc9 (Breast cancer);
5. Antigens which decrease chemotherapeutic efficacy of treatments (example); (CA Ant. Chem.): including but not limited to,
   a. interleukin-6.

The albumin-antibody will be directed towards facilitating removal of the targeted CA antigen(s): CA Ant. Aug., CA Ant. T., CA Ant. ST, CA Ant. Sp., CA Ant. Chem. After the removal of the CA antigen(s), the cleansed body fluid will be returned to the patient. The frequency of treatment and the specifically targeted CA antigen(s) to be removed would depend on the underlying symptomatology and pathology of the patient, and would be determined by the patient's physician. The article used in performing the method includes two-stages. The first stage includes a treatment chamber for addition of an antibody with an attached albumin moiety, which is added to the body fluid. A second stage receives the treated blood and/or CSF and includes a unit for removing the treatment.

The method includes providing a dialysis or other filtering machine with a first stage and a second stage, and sequentially passing the extracorporeal body fluid through the first and second stages. The body fluid is removed from the patient using standard procedure. The first stage applies a treatment using an antibody which was has attached to it an albumin moiety (or alternatively, a moiety which allows for the efficacious dialysis or removal by other techniques of the antibody-albumin-CA antigen), for the treatment of the body fluid. The second stage substantially removes the treatment. The purified body fluid (body fluid with removed targeted CA antigen: CA Ant. Aug., CA Ant. T., CA Ant. ST, CA Ant. Sp., CA Ant. Chem.)—is then tested for the efficacy of removal of the CA antigen and returned to the patient.

An alternative methodology of the present intervention would utilize a designer antibody with an attached macromolecular moiety instead of an albumin moiety. In embodiments, the macromolecular moiety attached to the antibody would be about 1.000 mm to 0.005 mm in diameter. The antibody-macromolecular moiety-targeted antigen complex would then be blocked from reentering the patient's body fluid circulation by using a series of screen filters or micro-screens which define openings with diameters less than the diameter of the designer antibody-macromolecular moiety. In one example, the openings are 50% to 99% of the size of the moiety. Conveniently, the openings will have diameters of at least 25micro meters in order to allow for the passage and return to circulation of the non-pathologic inducing body fluid constituents.

Alternatively, the target CA antigens may be captured by utilizing antibody microarrays which contain antibodies to targeted CA antigens. The antibody microarrays are composed of millions of identical monoclonal antibodies attached at high density on glass or plastic slides. After sufficient extracorporeal exposure of the targeted CA antigens to the antibody microarrays, the antibody microarrays-targeted CA antigens may be disposed of, utilizing standard medical practice.

Another alternative methodology of the present intervention comprises removing one or more of the targeted cancer antigens from the body fluid by utilizing a designer antibody containing an iron (Fe) moiety. This will then create an Fe-Antibody-Antigen complex. This iron containing complex may then be efficaciously removed utilizing a strong, localized magnetic force field.

The invention can also be used in combination with other therapies including, for example, Kanzius radiofrequency (RF) therapy as described in U.S. Pat. No. 7,510,555 and U.S. Pat. No. 7,627,381 which are hereby incorporated by reference. Kanzius therapy uses nanoparticles and RF radiation to induce hyperthermia in cancer cells.

The invention and Kanzius therapy are synergistic. Alone, Kanzius therapy can cause multiple infarctions in major organs leading to blindness, heart, attacks, and renal failure. Performing Kanzius therapy extracorporeally avoids these morbidities. Additionally, much higher levels of RF can be used. The invention can include a treatment comprising Kanzius therapy. Nanoparticle residue of the Kanzius therapy and cellular and/or pathogen debris can be substantially removed from the blood in the second stage. Reducing the residue and debris returned to a patient's vascular system can reduce deleterious vascular cascades such as coagulation and inflammation, which are further causes of patient morbidity.

Advantageously, a physician can use magnetic resonance angiography (MRA) or magnetic resonance venography (MRV) to determine the arterial and venous blood vessels to and from a tumor. These techniques can identify the blood vessels from which the extracorporeal blood can be extracted and into which the treated blood can be returned.

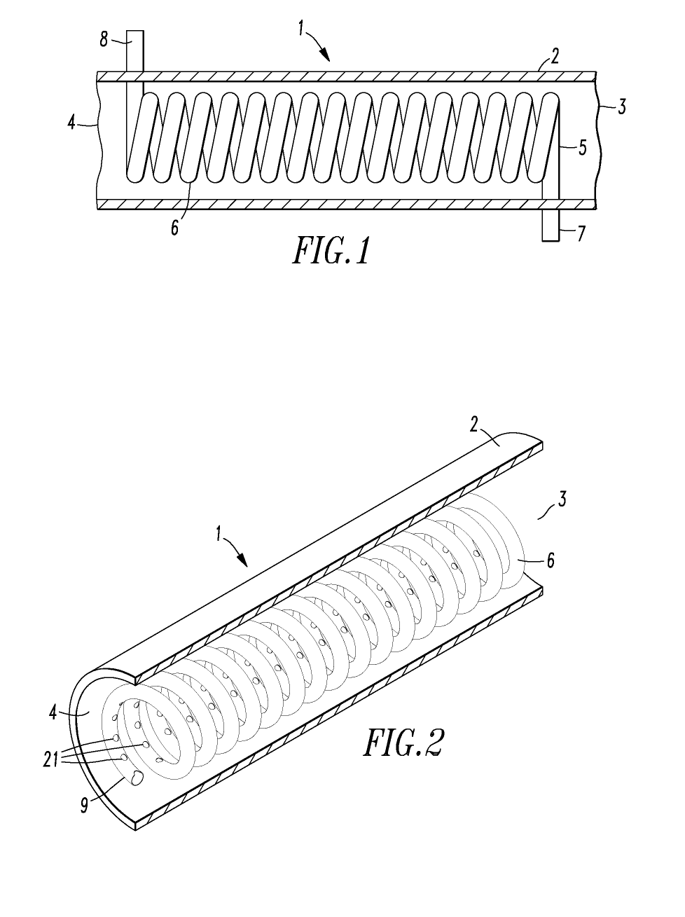

The device of the invention includes a first stage and a second stage. The first stage permits treatment of an antibody with an attached albumin moiety. The treatment targets the CA antigen(s) specifically exacerbating the pathologic condition. The second stage includes substantial removal of the treatment from the extracorporeal body fluid bodily fluid. As shown in FIG. 1, the first stage 1 can include an exterior wall 2 that defines a treatment chamber 5. The treatment conveniently can be applied in the treatment chamber 5. Residence times of the body fluid can be altered by changing the dimensions of the treatment chamber, or by using a dialysis vacuum pump. Body fluid enters the inlet 3, passes through the treatment chamber 5, and exits the outlet 4. In embodiments, the treatment of an antibody with an attached albumin moiety targeting the CA antigen(s) can be applied from a delivery tube 6 located within the treatment chamber 5. An interior wall 9 defines the delivery tube 6. The delivery tube 6 can include at least one lead 7,8. The lead 7, 8 can deliver the treatment to the treatment chamber 5. Conveniently, the delivery tubes 6 will, have a high contact surface area with the blood and/or CSF. In embodiments and as shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 in order to effect a desired concentration of designer and bodies in the body fluid. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic. The antibody with attached albumin moiety, targeting the CA antigen(s) can be delivered in a concurrent or counter-current mode with reference to the body fluid. In counter current mode, the body fluid enters the treatment chamber 5 at the inlet 3. The designer antibody can enter through a first lead 8 near the outlet 4 of the treatment chamber 5. The blood and/or CSF then passes to the outlet 4 and the designer antibodies pass to the second lead near the inlet 3. The removal module of the second stage substantially removes the designer antibodies-CA antigen molecular compound from the body fluid.

The second stage can include a filter, such as a dialysis machine, which is known to one skilled in the art. The second stage can include a molecular filter. For example, molecular adsorbents recirculating system (MARS), which may be compatible and/or synergistic with dialysis equipment. MARS technology can be used to remove small to average sized molecules from the body fluid. Artificial liver filtration presently uses this technique.

The method can include a plurality of steps for removing the targeted CA antigen(s). A first step can include directing a first antibody against the targeted antigen. A second step can include a second antibody. The second antibody can be conjugated with albumin, or alternatively another moiety which allows for efficacious dialysis or filtering of the antibody-CA antigen from the body fluid. The second antibody or antibody-albumen complex combines with the first antibody forming an antibody-antibody-moiety complex. A third step is then used to remove the complex from the blood and/or CSF. This removal is enabled by using dialysis and/or MARS. The purified body fluid is then returned to the patient.

In practice, a portion of the purified body fluid can be tested to ensure a sufficient portion of the targeted CA antigen(s) have been successfully removed from the body fluid. Testing can determine the length of treatment and evaluate the efficacy of the sequential dialysis methodology in removing the targeted CA antigen(s) and suggest the need for further treatment. Body fluid with an unacceptably large concentrations of complex, remaining can then be retreated and refiltered before returning the body fluid to the patient.

In embodiments, the second stage to remove the antibody-moiety-targeted CA antigen complex from the body fluid can be accomplished by various techniques including, for example, mechanical filtration and/or chemical filtration such as dialysis, filtering based on molecular size, protein binding, solubility, chemical reactivity, and combinations thereof. For example, a filter can include a molecular sieve, such as zeolite, or porous membranes mat capture complexes comprising molecules above a certain size. Membranes can comprise polyacrylonitrile, polysulfone, poly amides, cellulose, cellulose acetate, polyacrylates, polymethylmethacrylates, and combinations thereof. Increasing the flow rate or diasylate flow rate can increase the rate of removal of the antibody with attached albumin moiety targeting the CA antigen(s) such as CA Ant. Aug., CA Ant. T., CA Ant. ST, CA Ant. Sp., CA Ant. Chem.

Further techniques can include continuous renal replacement, therapy (CRRT) which can remove large quantities of filterable molecules from the extracorporeal body fluid, CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venoveaous high flux hemodialysis. The sieving coefficient (SC) is the ratio of the molecular concentration in the filtrate to the incoming CSF. A SC close to zero implies that the moiety-antibody-targeted antigen complex will not pass through the filter. A filtration rate of 50 ml per minute is generally satisfactory. Other methods of increasing the removability of the antibody-targeted antigen moiety include the use of temporary acidification of the body fluid extracorporeally using organic acids to compete with protein binding sites.

While the foregoing has been set forth in considerable detail, it is to be understood that the drawings and detailed, embodiments are presented, for elucidation and not limitation. Design variations, especially in matters of shape, size and arrangements of parts may be made but are within the principles of the invention. Those skilled in the art will realize that such changes or modifications of the invention or combinations of elements, variations, equivalents or improvements therein are still within the scope of the invention as defined in the appended claims.

1. A method for treating an extracorporeal body fluid comprising at least one CA antigen, the method characterized by:
a) combining a first antibody with the CA antigen in the extracorporeal body fluid to produce an antibody-CA antigen moiety; and
b) removing the antibody-CA antigen moiety from the extracorporeal body fluid.

2. The method of claim 1, wherein the CA antigen is selected from a group consisting of Angiogenesis (CA Ant. Ang.), Tumorigenesis (CA Ant. T.), Signal Transducer (CA Ant. ST), carcinoma specific antigens (CA Ant. Sp.), antigens which decrease chemotherapeutic efficacy (CA Ant. Chem.), and combinations thereof.

3. The method of claim 1, characterized by removing the antibody-CA antigen moiety includes irradiation, magnetism, mechanical filtering, chemical filtering, and combinations thereof.

4. The method of claim 1, further characterized by conjugating the antibody-CA antigen with albumin thereby forming an albumin-antibody-CA antigen compound.

5. The method of claim 1 further characterized by testing the extracorporeal body fluid for efficacy of removing the antibody-CA antigen moiety.

6. The method of claim 1 further characterized by removing a body fluid from a patient to produce the extracorporeal body fluid and returning the extracorporeal body fluid to the patient after treating the extracorporeal body fluid.

7. The method of claim 1, characterized by combining the first antibody with the CA antigen in a first stage, passing the extracorporeal body fluid to a second stage, and removing the antibody-CA antigen moiety from the body fluid in the second stage.

8. The method of claim 7, characterized by providing a filtering machine comprising the first stage and the second stage, and sequentially passing the extracorporeal body fluid through the first and second stages.

9. The method of claim 7, characterized by conjugating the antibody-CA antigen with albumin in the first stage, thereby forming an albumin-antibody-CA antigen compound.

10. The method of claim 1, characterized by conjugating the antibody-CA antigen with a designer antibody comprising an attached macromolecular moiety, thereby forming an antibody-macromolecular moiety-targeted antigen complex having a diameter.

11. The method of claim 10, characterized by the diameter of the antibody-macromolecular moiety-targeted antigen complex being from about 0.905 mm to 1,000 mm.

12. The method of claim 10, characterized by removing the antibody-macromolecular moiety-targeted antigen complex by filtering through at least one screen filter defining a plurality of openings having opening diameters less than the diameter of the antibody macromolecular moiety-targeted antigen complex.

13. The method of claim 12, characterized by the opening diameters being 50% to 99% of the diameter of the antibody-macromolecular moiety-targeted antigen complex.

14. The method of claim 12, characterized by the opening diameters being at least 25 micrometers.

15. The method of claim 1, characterized by the first antibody being fixed to an antibody microarray, whereby removing the antibody-CA antigen moiety from the extracorporeal body fluid comprises fixing the antibody-CA antigen moiety to the microarray.

16. The method of claim 1, characterized by combining the antibody-CA antigen moiety with at least one antibody containing iron, thereby forming an Fe-Antibody-Antigen complex, and removing the Fe-Antibody-Antigen complex using a strong, localized magnetic field.

17. The method of claim 1, characterized by removing the antibody-CA antigen moiety using Kanzius radiofrequency (RF) therapy and removing residue of the Kanzius radiofrequency (RF) therapy from the extracorporeal body fluid.

18. The method of claim 1, characterized by removing the antibody-CA antigen moiety using a molecular filter.

19. The method of claim 1, characterized by removing the antibody-CA antigen moiety using a molecular sieve comprising a material selected from a group consisting of zeolite, polyacrylonitrile, polysulfone, polyamide, cellulose, cellulose acetate, polyacrylate, polymethylmethacrylate, and combinations thereof.

20. The method of claim 1, further characterized by retreating the extracorporeal body fluid if an unacceptably large concentration of antibody-CA antigen moiety remains in the extracorporeal body fluid.
